(19) United States
(12) Patent Application Publication
(10) Pub. No.: US 2025/0036738 A1
(43) Pub. Date: Jan. 30, 2025

(54) ELECTRONIC DEVICE AND CONTROLLING METHOD OF ELECTRONIC DEVICE

(71) Applicant: Samsung Electronics Co., Ltd., Suwon-si (KR)

(72) Inventors: Dmytro PROGONOV, Kyiv (UA); Andrii ASTRAKHANTSEV, Kyiv (UA); Oleksandra SOKOL, Kyiv (UA); Dmytro DEGTIAROV, Kyiv (UA); Heorhii NAUMENKO, Kyiv (UA); Ivan SHAPOVAL, Kyiv (UA); Kyrylo ROMANII, Kyiv (UA); Maksym ZEMLIANOI, Kyiv (UA); Viacheslav DERKACH, Kyiv (UA); Yevgen ROMANKO, Kyiv (UA)

(21) Appl. No.: 18/918,910

(22) Filed: Oct. 17, 2024

Related U.S. Application Data
(63) Continuation of application No. PCT/KR2024/008997, filed on Jun. 27, 2024.

(30) Foreign Application Priority Data
Jul. 21, 2023 (KR) 10-2023-0095599

(51) Int. Cl. G06F 21/32

(52) U.S. Cl. CPC G06F 21/32

(57) ABSTRACT

An electronic device and a method of controlling the electronic device are provided. The electronic device includes at least one motion sensor, at least one biometric sensor, memory storing one or more computer programs, and one or more processors communicatively coupled to the at least one motion sensor, the at least one biometric sensor, and the memory, wherein the one or more computer programs include computer-executable instructions that, when executed by the one or more processors cause the electronic device to, based on receiving a request for user authentication, acquire a first signal through the at least one motion sensor, and acquire a second signal through the at least one biometric sensor, acquire motion information indicating a type of a user's motion based on the first signal, acquire biometric information indicating a biometric feature of a user according to the motion of the user based on the second signal, and input the motion information and the biometric information into a user authentication model, and perform the user authentication according to whether the type of the motion and the biometric feature match authentication information.

### CROSS-REFERENCE TO RELATED APPLICATION(S)

This application is a continuation application, claiming priority under § 365(c), of an International application No. PCT/KR2024/008997, filed on Jun. 27, 2024, which is based on and claims the benefit of a Korean patent application number 10-2023-0095599, filed on Jul. 21, 2023, in the Korean Intellectual Property Office, the disclosure of which is incorporated by reference herein in its entirety.

### TECHNICAL FIELD

The disclosure relates to an electronic device and a controlling method of the electronic device. More particularly, the disclosure relates to an electronic device that can perform user authentication, and a controlling method of the electronic device.

### BACKGROUND ART

Recently, risk regarding security is increasing as portability and connectivity with another device of an electronic device have been reinforced. For example, recent portable electronic devices including a smartphone and a smart watch are providing a function which enables easy access to information stored in various external devices connected to the electronic devices as well as the electronic devices, and accordingly, risk that sensitive information related to a user's private life or health, or the like, may be leaked is increasing.

Also, connection with an e-mail and social media can be performed through an electronic device, and further, various functions related to finance and payment can be performed, and accordingly, risk regarding security is greatly increasing.

For securing security of an electronic device, technologies for performing user authentication by using fingerprint recognition, iris recognition, face recognition, or the like, instead of a password input of the related art are being developed, but regarding the existing technologies, a limitation that the technologies still cannot sufficiently cope with technics that threaten security, such as spoofing or phishing, or the like, is being pointed out.

Also, in the case of the existing technologies, there are limitations that there is a restriction on the size of a display like a smart watch, or the technologies cannot be applied to a device wherein it is difficult to mount a separate sensor, such as a high performance camera, a fingerprint recognition sensor, or the like.

The above information is presented as background information only to assist with an understanding of the disclosure. No determination has been made, and no assertion is made, as to whether any of the above might be applicable as prior art with regard to the disclosure.

### DISCLOSURE

### Technical Solution

Aspects of the disclosure are to address at least the above-mentioned problems and/or disadvantages and to provide at least the advantages described below. Accordingly, an aspect of the disclosure is to provide an electronic device that can perform correct and effective user authentication, and a controlling method of the electronic device.

Additional aspects will be set forth in part in the description which follows and, in part, will be apparent from the description, or may be learned by practice of the presented embodiments.

In accordance with an aspect of the disclosure, an electronic device is provided. The electronic device includes at least one motion sensor, at least one biometric sensor, memory storing one or more computer programs, and one or more processors communicatively coupled to the at least one motion sensor, the at least one biometric sensor, and the memory, wherein the one or more computer programs include computer-executable instructions that, when executed by the one or more processors, based on receiving a request for user authentication, acquire a first signal through the at least one motion sensor, acquire a second signal through the at least one biometric sensor, acquire motion information indicating a type of a user's motion based on the first signal, acquire biometric information indicating a biometric feature of a user according to the motion of the user based on the second signal, and input the motion information and the biometric information into a user authentication model, and perform the user authentication according to whether the type of the motion and the biometric feature match authentication information.

Here, the one or more computer programs further include computer-executable instructions that, when executed by the one or more processors, cause the electronic device to, based on identifying that the user is a registered user as a result that the user authentication was performed, release locking of the electronic device, and update the authentication information based on the motion information and the biometric information.

Meanwhile, the one or more computer programs further include computer-executable instructions that, when executed by the one or more processors, cause the electronic device to, based on the first signal starting to be received through the at least one motion sensor, acquire a second signal through the at least one biometric sensor.

Meanwhile, the at least one biometric sensor includes a bone conduction sensor, and the biometric information includes information on an audio feature transmitted from at least one of a bone or a joint of the user according to the motion of the user.

Meanwhile, the at least one biometric sensor includes a bioimpedance sensor, and the biometric information includes information on an electrical feature transmitted from at least one of the muscle, the tissue, or the joint of the user according to the motion of the user.

Meanwhile, the at least one biometric sensor includes a heartbeat sensor, and the biometric information includes information on a heartbeat feature transmitted from the heart of the user while the motion of the user is being performed.

Here, the one or more computer programs further include computer-executable instructions that, when executed by the one or more processors, cause the electronic device to, acquire stress information indicating the stress level of the user based on the biometric information, and the user authentication model performs the user authentication according to whether the stress level, the type of the motion, and the heartbeat feature match the authentication information.

Meanwhile, the one or more computer programs further include computer-executable instructions that, when executed by the one or more processors, cause the electronic device to, perform pre-processing for removing at least one of noises or trends of the first signal and the second signal, and acquire the motion information and the biometric information based on the pre-processed first signal and the pre-processed second signal.

Meanwhile, the electronic device includes a wearing part, and one or more sensors included in the at least one motion sensor and the at least one biometric sensor is coupled to the wearing part.

Meanwhile, the electronic device further includes a communicator, and the one or more computer programs further include computer-executable instructions that, when executed by the one or more processors, cause the electronic device to, based on identifying that the user is an unregistered user as a result that the user authentication was performed, maintain locking of the electronic device, and control the communicator to transmit a message for guiding that authentication by an unregistered user was performed to an external device connected to the electronic device.

Meanwhile, the electronic device further includes an outputter, and the one or more computer programs further include computer-executable instructions that, when executed by the one or more processors, cause the electronic device to, based on identifying that the user is an unregistered user as a result that the user authentication was performed, maintain locking of the electronic device, and control the outputter to output a guide message for requesting additional authentication.

In accordance with another aspect of the disclosure, a method of controlling an electronic device is provided. The method includes based on receiving a request for user authentication, acquiring a first signal through at least one motion sensor, and acquiring a second signal through at least one biometric sensor, acquiring motion information indicating a type of a user's motion based on the first signal, acquiring biometric information indicating a biometric feature of a user according to the motion of the user based on the second signal, and inputting the motion information and the biometric information into a user authentication model, and performing the user authentication according to whether the type of the motion and the biometric feature match authentication information.

Here, the method of controlling the electronic device further includes based on identifying that the user is a registered user as a result that the user authentication was performed, releasing locking of the electronic device, and updating the authentication information based on the motion information and the biometric information.

Meanwhile, the acquiring of the second signal includes, based on the first signal starting to be received through the at least one motion sensor, acquiring a second signal through the at least one biometric sensor.

Meanwhile, the at least one biometric sensor includes a bone conduction sensor, and the biometric information may include information on an audio feature transmitted from at least one of a bone or a joint of the user according to the motion of the user.

Meanwhile, the at least one biometric sensor includes a bioimpedance sensor, and the biometric information may include information on an electrical feature transmitted from at least one of the muscle, the tissue, or the joint of the user according to the motion of the user.

Meanwhile, the at least one biometric sensor includes a heartbeat sensor, and the biometric information may include information on a heartbeat feature transmitted from the heart of the user while the motion of the user is being performed.

Here, the method of controlling an electronic device further includes acquiring stress information indicating the stress level of the user based on the biometric information, and the user authentication model may perform the user authentication according to whether the stress level, the type of the motion, and the heartbeat feature match the authentication information.

Meanwhile, the method of controlling an electronic device further includes performing pre-processing for removing at least one of noises or trends of the first signal and the second signal, and acquiring the motion information and the biometric information based on the pre-processed first signal and the pre-processed second signal.

Meanwhile, the method of controlling an electronic device further includes, based on identifying that the user is an unregistered user as a result that the user authentication was performed, maintaining locking of the electronic device, transmitting a message for guiding that authentication by an unregistered user was performed to an external device connected to the electronic device, and outputting a guide message for requesting additional authentication.

In accordance with another aspect of the disclosure, one or more non-transitory computer readable storage media storing computer-executable instructions that, when executed by one or more processors of an electronic device, cause the electronic device to perform operations are provided. The operations include based on receiving a request for user authentication, acquiring a first signal through at least one motion sensor, and acquiring a second signal through at least one biometric sensor, acquiring motion information indicating a type of a user's motion based on the first signal, acquiring biometric information indicating a biometric feature of a user according to the motion of the user based on the second signal, and inputting the motion information and the biometric information into a user authentication model, and performing the user authentication according to whether the type of the motion and the biometric feature match authentication information.

Other aspects, advantages, and salient features of the disclosure will become apparent to those skilled in the art from the following detailed description, which, taken in conjunction with the annexed drawings, discloses various embodiments of the disclosure.

### DESCRIPTION OF DRAWINGS

The above and other aspects, features, and advantages of certain embodiments of the disclosure will be more apparent from the following description taken in conjunction with the accompanying drawings, in which:

FIG. 1 is a block diagram schematically illustrating a configuration of an electronic device according to an embodiment of the disclosure;

FIG. 2 is a block diagram illustrating a plurality of modules according to an embodiment of the disclosure;

FIG. 3 is a block diagram illustrating a plurality of modules according to an embodiment of the disclosure;

FIG. 4 is a block diagram illustrating a configuration of an electronic device according to an embodiment of the disclosure;

FIGS. 5 and 6 are flowcharts illustrating a controlling method of an electronic device according to various embodiments of the disclosure; and

FIG. 7 is a flowchart illustrating a controlling method of an electronic device according to an embodiment of the disclosure.

The same reference numerals are used to represent the same elements throughout the drawings.

### MODE FOR INVENTION

The following description with reference to the accompanying drawings is provided to assist in a comprehensive understanding various embodiments of the disclosure as defined by the claims and their equivalents. It includes various specific details to assist in that understanding but these are to be regarded as merely exemplary Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Also, in describing the disclosure, in case it is determined that detailed explanation of related known functions or characteristics may unnecessarily confuse the gist of the disclosure, the detailed explanation will be omitted.

In addition, the embodiments described below may be modified in various different forms, and the scope of the technical idea of the disclosure is not limited to the embodiments below. Rather, these embodiments are provided to make the disclosure more sufficient and complete, and to fully convey the technical idea of the disclosure to those skilled in the art.

In addition, the terms used in the disclosure are used only to explain specific embodiments of the disclosure, and are not intended to limit the scope of the disclosure.

In addition, in the disclosure, expressions, such as “have,” “may have,” “include,” and “may include” denote the existence of such characteristics (e.g., elements, such as numbers, functions, operations, and components), and do not exclude the existence of additional characteristics.

Further, in the disclosure, the expressions “A or B,” “at least one of A and/or B,” or “one or more of A and/or B” and the like may include all possible combinations of the listed items. For example, “A or B,” “at least one of A and B,” or “at least one of A or B” may refer to all of the following cases: (1) including at least one A, (2) including at least one B, or (3) including at least one A and at least one B.

Also, the expressions “first,” “second,” and the like used in the disclosure may describe various elements regardless of any order and/or degree of importance. Also, such expressions are used only to distinguish one element from another element, and are not intended to limit the elements.

Meanwhile, the description in the disclosure that one element (e.g., a first element) is “(operatively or communicatively) coupled with/to” or “connected to” another element (e.g., a second element) should be interpreted to include both the case where the one element is directly coupled to the other element, and the case where the one element is coupled to the other element through still another element (e.g., a third element).

In contrast, the description that one element (e.g., a first element) is “directly coupled” or “directly connected” to another element (e.g., a second element) can be interpreted to mean that still another element (e.g., a third element) does not exist between the one element and the other element.

Also, the expression “configured to” used in the disclosure may be interchangeably used with other expressions, such as “suitable for,” “having the capacity to,” “designed to,” “adapted to,” “made to,” and “capable of,” depending on cases. Meanwhile, the term “configured to” may not necessarily mean that a device is “specifically designed to” in terms of hardware.

Instead, under some circumstances, the expression “a device configured to” may mean that the device “is capable of” performing an operation together with another device or component. For example, the phrase “a processor configured to perform A, B, and C” may mean a dedicated processor (e.g., an embedded processor) for performing the corresponding operations, or a generic-purpose processor (e.g., a CPU or an application processor) that can perform the corresponding operations by executing one or more software programs stored in memory.

In addition, in the embodiments of the disclosure, ‘a module’ or ‘a unit’ may perform at least one function or operation, and may be implemented as hardware or software, or as a combination of hardware and software. Also, a plurality of ‘modules’ or ‘units’ may be integrated into at least one module and implemented as at least one processor, excluding ‘a module’ or ‘a unit’ that needs to be implemented as specific hardware.

Meanwhile, various elements and areas in the drawings were illustrated schematically. Accordingly, the technical idea of the disclosure is not limited by the relative sizes or intervals illustrated in the accompanying drawings.

It should be appreciated that the blocks in each flowchart and combinations of the flowcharts may be performed by one or more computer programs which include computer-executable instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g., a central processing unit (CPU)), a communication processor (CP, e.g., a modem), a graphical processing unit (GPU), a neural processing unit (NPU) (e.g., an artificial intelligence (AI) chip), a wireless-fidelity (Wi-Fi) chip, a Bluetooth™ chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an IC, or the like.

Hereinafter, embodiments according to the disclosure will be described with reference to the accompanying drawings, such that those having ordinary skill in the art to which the disclosure belongs can easily carry out the disclosure.

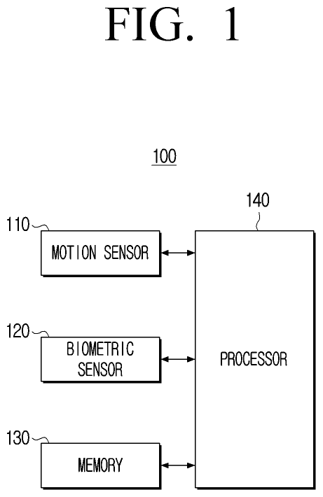

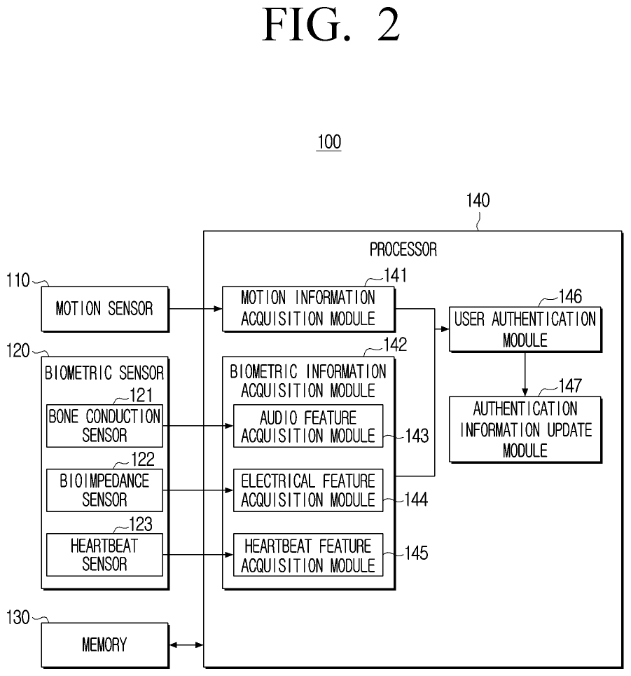

FIG. 1 is a block diagram schematically illustrating a configuration of an electronic device according to an embodiment of the disclosure. FIG. 2 is a block diagram illustrating a plurality of modules according to an embodiment of the disclosure. Hereinafter, explanation will be described with reference to FIGS. 1 and 2 together.

Referring to FIG. 1, ‘an electronic device 100’ according to the disclosure refers to a device that can perform user authentication. In particular, the electronic device 100 may be a wearable device that can be worn on a user's body. For example, the electronic device 100 may be implemented as one of a smart watch, a smart band, smart glasses, a head-mounted display (HMD), or smart clothing. However, there is no specific limitation on the type of the electronic device 100.

‘User authentication’ refers to a process of identifying whether a specific user is a legitimate user of the electronic device 100. For example, user authentication may include a process of identifying whether a user who is a subject of authentication is a user who was registered as the user of the electronic device 100, or is a user who has authority to access applications or information provided by the electronic device 100. The term ‘user authentication’ may be replaced by terms, such as ‘user verification’ or ‘user identification,’ or the like.

Referring to FIG. 1, the electronic device 100 may include a motion sensor 110, a biometric sensor 120, memory 130, and a processor 140.

‘The motion sensor 110’ refers to a sensor that can detect a signal according to a motion of the electronic device 100. The electronic device 100 may include at least one motion sensor 110. For example, the at least one motion sensor 110 may include at least one of an accelerometer or a gyroscope, but is not limited thereto.

The at least one motion sensor 110 may acquire a signal indicating a motion of the electronic device 100. For example, the accelerometer may receive a signal indicating a change rate of the electronic device 100 that changes according to time, and the gyroscope may receive a signal indicating the rotation speed and the direction of the electronic device 100. Hereinafter, a signal acquired through the at least one motion sensor 110 is referred to as ‘a first signal,’ and the term ‘a first signal’ may be replaced by a term, such as ‘a motion signal,’ or the like.

Meanwhile, in case the electronic device 100 is implemented as a wearable device that can be worn on a user's body, a motion of the electronic device 100 may correspond to a motion of the user. Accordingly, hereinafter, the term ‘a motion of the user’ and the term ‘a motion of the electronic device 100’ may be replaced by each other.

Hereinafter, the term ‘a motion’ is used as a term for generally referring to behaviors that cause a change of the location of the user or the electronic device 100 according to time. Accordingly, the term ‘a motion’ may be replaced by the term ‘an action’ or ‘a movement.’ Also, the term ‘a motion’ may be used as meaning including ‘a posture’ which means a static state of the user's body after a motion was performed or ‘an activity’ which means a collection of a series of motions in a broad sense.

‘The biometric sensor 120’ refers to a sensor that can detect a signal related to biometric information of the user. The electronic device 100 may include at least one biometric sensor 120, and the at least one biometric sensor 120 may include at least one of a bone conduction sensor 121, a bioimpedance sensor 122, or a heartbeat sensor 123.

The bone conduction sensor 121 may receive a sound signal according to vibration of at least one of the bone or the joint of the user. The sound signal received through the bone conduction sensor 121 may indicate the feature of a sound transmitted through at least one of the bone or the joint of the user.

The bone conduction sensor 121 may be replaced by an acoustic sensor or a microphone, but an acoustic sensor or a microphone is generally optimized for detecting a sound transmitted through the air, and accordingly, in case an acoustic sensor or a microphone is used in place of the bone conduction sensor 121, it would be preferable that an acoustic sensor or a microphone optimize for acquiring the feature of a sound transmitted from at least one of the bone or the joint is used.

The bioimpedance sensor 122 may transmit an electric signal to a component of the user's body, and receive an electric signal indicating resistance and reactance of the user's muscle, tissue, joint, or the like, as a response to the transmitted signal. The signal received through the bioimpedance sensor 122 may indicate a physiological feature, such as the body fat amount, the body water amount, the muscle amount, the liquid amount inside the cells, or the like, of the user.

The heartbeat sensor 123 may receive a heartbeat signal (HBS) that is generated according to contraction and relaxation of the user's heart. Also, the heartbeat sensor 123 may receive an electrocardiogram (ECG) signal indicating an electrical activity of the heart and a heart rate variability (HRV) signal indicating variability of the heart rate. A signal received through the heartbeat sensor 123 may indicate a feature related to the health of the heart, the exercise result, the stress management, or the like, of the user.

Other than the above, the biometric sensor 120 may include various sensors, such as a photoplethysmography (PPG) sensor that can measure a pulse and a blood flow and measure oxygen saturation and heart rates by detecting a change of light reflected on the surface of the skin and an electrocardiogram sensor, or the like.

Hereinafter, a signal acquired through the at least one biometric sensor 120 will be referred to as ‘a second signal’ by distinguishing it from the first signal which is a signal acquired through the at least one motion sensor 110, and the term ‘a second signal’ may be replaced by a term, such as ‘a biometric signal.’

In the memory 130, at least one instruction regarding the electronic device 100 may be stored. Also, in the memory 130, an operating system (O/S) for driving the electronic device 100 may be stored. In addition, in the memory 130, various types of software programs or applications for making the electronic device 100 operate according to the various embodiments of the disclosure may be stored. Further, the memory 130 may include a semiconductor memory, such as a flash memory, or the like, or a magnetic storage medium, such as a hard disk, or the like.

Specifically, in the memory 130, various types of software modules for the electronic device 100 to operate according to the various embodiments of the disclosure may be stored, and the processor 140 may control the operations of the electronic device 100 by executing the various types of software modules stored in the memory 130. For example, the memory 130 may be accessed by the processor 140, and reading/recording/correction/deletion/update, or the like, of data by the processor 140 may be performed.

Meanwhile, in the disclosure, the term memory 130 may be used as meaning including the memory 130, read only memory (ROM) and random access memory (RAM) inside the processor 140, or memory card (e.g., a micro secure digital (SD) card, a memory stick) mounted on the electronic device 100.

More particularly, in the memory 130, various kinds of information, such as information on a user authentication model, authentication information for the user, information on a plurality of modules, motion information, biometric information, stress information, information on an algorithm for processing and analyzing signals received through the at least one motion sensor 110 and the at least one biometric sensor 120, or the like, may be stored.

Other than the above, various kinds of information necessary within a range for achieving the purpose of the disclosure may be stored in the memory 130, and the information stored in the memory 130 may be renewed as information is received from an external device or is input by the user.

The processor 140 controls the overall operations of the electronic device 100. Specifically, the processor 140 may be connected with the components of the electronic device 100 including the motion sensor 110, the biometric sensor 120, and the memory 130, and may control the overall operations of the electronic device 100 by executing at least one instruction stored in the memory 130 as described above.

The processor 140 may be implemented by various methods. For example, the processor 140 may be implemented as at least one of an application specific integrated circuit (ASIC), an embedded processor, a microprocessor, a hardware control logic, a hardware finite state machine (FSM), or a digital signal processor (DSP). Meanwhile, in the disclosure, the term processor 140 may be used as meaning including a central processing unit (CPU), a graphics processing unit (GPU), and a micro processor unit (MPU), or the like.

In particular, the processor 140 may perform user authentication based on signals received through various types of sensors. The processor 140 may perform various processing processes related to user authentication by using a plurality of modules as illustrated in FIG. 2. The plurality of modules may consist of a hardware module or a software module, and at least one module among the plurality of modules may be implemented through a neural network.

Meanwhile, hereinafter, explanation will be described on the premise of a case wherein all of the plurality of modules are implemented as on-devices through the processor 140 of the electronic device 100. However, it is obvious that at least one of the plurality of modules can be implemented through an external device or a server.

Referring to FIG. 2, the plurality of modules may include a motion information acquisition module 141, a biometric information acquisition module 142, a user authentication module 146, and an authentication information update module 147. Also, the biometric information acquisition module 142 may include an audio feature acquisition module 143, an electrical feature acquisition module 144, and a heartbeat feature acquisition module 145. Hereinafter, various embodiments of the disclosure implemented through the processor 140 and the plurality of modules will be described.

When a request for user authentication is received, the processor 140 may acquire a first signal through the at least one motion sensor 110, and acquire a second signal through the at least one biometric sensor 120.

‘A request for user authentication’ may be received based on a user input for initiating user authentication, or received based on generation of an event which was defined in advance that user authentication is necessary. For example, a request for user authentication may be received based on the user's touch input for releasing locking of the electronic device 100 which is in a locked state. Also, a request for user authentication may be received based on generation of an event which was defined in advance that user authentication is necessary, such as receipt of a phone call, checking of a message, proceeding of payment, or the like, in the electronic device 100.

According to one or more embodiments of the disclosure, if the first signal is received through the at least one motion sensor 110, the processor 140 may acquire the second signal through the at least one biometric sensor 120. For example, the processor 140 may receive the second signal through the at least one biometric sensor 120 during a predetermined period after the first signal started to be received through the at least one motion sensor 110 according to a motion of the user (e.g., from the time point when receipt of the first signal was initiated to the time point when receipt of the first signal was completed). In other words, for detecting a change of the biometric information of the user according to a motion of the user, the processor 140 may receive the second signal through the at least one biometric sensor 120 while the motion of the user is being performed.

The first signal and the second signal may be received in analog or digital forms, and the processor 140 may acquire motion information and biometric information by performing various kinds of processing and analysis of various signals as will be described below regarding the received first signal and second signal.

According to one or more embodiments of the disclosure, the processor 140 may perform pre-processing for removing at least one of noises and/or trends of the first signal and/or the second signal (i.e., de-noising and de-trending), and acquire motion information and biometric information as will be described below based on the pre-processed first signal and the pre-processed second signal.

For example, the processor 140 may remove noises of the first signal and the second signal by calculating a moving average of the first signal and the second signal, or using a low-pass filter or a high-pass filter for the first signal and the second signal. Other than the above, a wavelet transformation, or the like, as will be described below may be used for removing noises of the first signal and the second signal. Also, the processor 140 may remove trends of the first signal and/or the second signal by using a polynomial approximation, a spline approximation, or an empirical mode decomposition (EMD), or using regression modeling for the first signal and the second signal.

The processor 140 may acquire motion information indicating the type of the user's motion based on the first signal. As illustrated in FIG. 2, if the first signal is acquired through the motion sensor 110, the processor 140 may acquire motion information indicating the type of the user's motion by inputting the first signal into the motion information acquisition module 141. ‘The motion information’ may be a vector or a tensor indicating the type of the user's motion, but is not limited thereto.

‘The motion information acquisition module 141’ refers to a module that can acquire motion information indicating the type of the user's motion by analyzing the first signal acquired through the motion sensor 110.

According to one or more embodiments of the disclosure, the motion information acquisition module 141 may extract statistical features for the first signal, and acquire the motion information based on the extracted statistical features. For example, the motion information acquisition module 141 may acquire the motion information by analyzing features, such as an average, a variance, a maximum value, a minimum value, a standard deviation, or the like, of the first signal by applying a statistical model, such as a gaussian model or a sparse model, or the like, to the first signal.

According to one or more embodiments of the disclosure, the motion information acquisition module 141 may extract frequency domain features for the first signal, and acquire the motion information based on the extracted frequency domain features. For example, the motion information acquisition module 141 may acquire a spectrogram corresponding to the first signal by applying a blackman-harris window and a short-time fourier transformation (STFT) for the first signal. The motion information acquisition module 141 may convert the acquired spectrogram into eight bits, and input the spectrogram converted into eight bits into a trained neural network model, and may thereby acquire information on the frequency domain features of the first signal. Then, the motion information acquisition module 141 may acquire motion information corresponding to the information on the frequency domain features for the first signal.

According to one or more embodiments of the disclosure, the motion information acquisition module 141 may acquire the motion information by combining the statistical features for the first signal and the frequency domain features for the first signal. For example, the motion information acquisition module 141 may acquire a first vector indicating statistical features for the first signal and a second vector indicating frequency domain features for the first signal, and perform operations, such as a concatenation, an average, or a weighted average, or the like, of the first vector and the second vector, and may thereby acquire a third vector comprehensively indicating the statistical features for the first signal and the frequency domain features for the first signal. Then, the motion information acquisition module 141 may acquire motion information corresponding to the third vector by inputting the acquired third vector into the neural network model.

Meanwhile, ‘acquiring motion information indicating the type of the user's motion’ may include a process of identifying which type among a plurality of predefined motion types the user's motion indicated by the feature of the first signal corresponds to. For example, the plurality of predefined motion types may include a swing motion, a grip motion, and a shake motion, or the like, and may include various types other than them.

The processor 140 may acquire biometric information indicating a biometric feature of the user according to a motion of the user based on the second signal. As illustrated in FIG. 2, if the second signal is acquired through the biometric sensor 120, the processor 140 may acquire biometric information indicating a biometric feature of the user according to a motion of the user by inputting the second signal into the biometric information acquisition module 142. ‘The biometric information’ may be a vector or a tensor indicating a biometric feature of the user, but is not limited thereto. ‘Biometric features’ refer to unique features of the individual user that can identify the user, and may include physical features and physiological features of the user.

‘The biometric information acquisition module 142’ refers to a module that can acquire biometric information indicating a biometric feature of the user according to a motion of the user by performing analysis for the second signal acquired through the biometric sensor 120. The biometric information acquisition module 142 may include an audio feature acquisition module 143, an electrical feature acquisition module 144, and a heartbeat feature acquisition module 145.

According to one or more embodiments of the disclosure, if the second signal is received through the bone conduction sensor 121, the audio feature acquisition module 143 may acquire a spectrogram corresponding to the first signal by applying a blackman-harris window and a short-time fourier transformation (STFT) as described above for the second signal (a sound signal). Here, the acquired spectrogram may be a mel spectrogram that was acquired by converting a frequency domain by modeling a person's auditory feature. The audio feature acquisition module 143 may input the acquired spectrogram into the trained neural network model, and acquire information on an audio feature transmitted from at least one of the bone or the joint of the user according to a motion of the user as a type of biometric information.

According to one or more embodiments of the disclosure, if the second signal is received through the bioimpedance sensor 122, the electrical feature acquisition module 144 may acquire a wavelet spectrum by applying a discrete wavelet transformation (DWT) for the second signal (an electric signal). Here, the discrete wavelet transformation refers to a technic for extracting a frequency and time information of a signal by decomposing a signal in a time-frequency domain and expressing it as a wavelet coefficient and a scale coefficient. The electrical feature acquisition module 144 may convert the acquired wavelet spectrum into eight bits, and input the spectrogram converted into eight bits into the trained neural network model, and acquire information on an electrical feature transmitted from at least one of the muscle, the tissue, or the joint of the user according to a motion of the user as a type of biometric information.

According to one or more embodiments of the disclosure, if the second signal is received through the heartbeat sensor 123, the heartbeat feature acquisition module 145 may detect R-peaks which are the main peaks of a heartbeat by applying a Pan-Tompkins algorithm to the second signal (a heartbeat signal), and divide the second signal according to intervals among the R peaks, and normalize the segments of the divided second signal by using an L1 or L2 norm. Afterwards, the heartbeat feature acquisition module 145 may extract a spectrum feature of the second signal for which normalization has been performed, and acquire a wavelet spectrum by applying a discrete wavelet transformation or a stationary wavelet transformation (SWT).

Meanwhile, the heartbeat feature acquisition module 145 may perform self-similarity feature extraction for the second signal, and acquire information on a fractal dimension indicating a repetitive pattern and complexity of the signal. Then, the heartbeat feature acquisition module 145 may apply a multifractal analysis (MFA) technic or a singular spectrum analysis (SSA) technic for the information regarding the fractal dimension, and acquire information on the fractal dimension for each of the various scales or the several components of the second signal.

The heartbeat feature acquisition module 145 may combine the wavelet spectrum and the information on the fractal dimension as described above, and acquire information on the heartbeat feature transmitted from the user's heart while a motion of the user is being performed. Here, combination may be performed based on operations, such as a concatenation, an average, or a weighted average, or the like, as described above.

The processor 140 may input motion information and biometric information into a user authentication model, and perform user authentication according to whether the type of the motion and the biometric feature match the authentication information. As illustrated in FIG. 2, if motion information is acquired through the motion information acquisition module 141, and biometric information is acquired through the biometric information acquisition module 142, the processor 140 may perform user authentication by inputting the motion information and the biometric information into the user authentication module 146. Here, ‘the user authentication module 146’ refers to a module that performs user authentication, and may include a user authentication model including a neural network.

‘The user authentication model’ refers to a neural network model trained to perform user authentication. The user authentication model may be trained to identify whether input motion information and biometric information match authentication information that was constructed in advance. Specifically, the processor 140 may acquire a third vector by combining the first vector corresponding to the motion information and the second vector corresponding to the biometric information, and input the acquired third vector into the user authentication model. When the third vector is input, the user authentication model may identify whether the feature indicated by the third vector matches the features of the authentication information constructed in advance, and output information regarding the identification result.

First, the user authentication model may be trained in advance so that it can recognize the plurality of predefined types of motions. The user authentication model trained in advance may be distributed to the user by being transmitted to the electronic device 100 or enabling the user to access the server providing the user authentication model, and a user registration process may proceed afterwards.

When the user registration process is initiated, the user may perform at least one motion (e.g., a shaking motion of shaking the electronic device 100) among the plurality of predefined types of motions. When at least one motion is performed, the processor 140 may acquire the first signal through the at least one motion sensor 110, and acquire the second signal through the at least one biometric sensor 120 while each of the at least one motion is being performed. The processor 140 may acquire motion information indicating the type of the user's motion based on the first signal, and acquire biometric information indicating the biometric feature of the user according to the motion of the user based on the second signal.

Afterwards, the processor 140 may match each of the plurality of types of motions and biometric information corresponding thereto, and train the user authentication model to generate authentication information. For example, regarding the user authentication model, a few-shot calibration may be performed to fit the biometric information of an individual user. Accordingly, ‘the authentication information’ may include information on the type of the motion performed by user during the user authentication process among the plurality of predefined types of motions and a biometric feature of the user corresponding thereto. The term ‘authentication information’ may be replaced by terms, such as ‘user authentication data’ or ‘a user authentication template,’ or the like.

Meanwhile, the feature that a type of a motion and a biometric feature ‘match’ the authentication information may include not only a case wherein the type of the motion and the biometric feature ‘coincide with’ the authentication information, but also a case wherein the type of the motion and the biometric feature are ‘similar to’ the authentication information. The expression that the type of the motion and the biometric feature ‘match’ the authentication information may be replaced by an expression that the type of the motion and the biometric feature ‘correspond to’ the authentication information, or the like.

According to one or more embodiments of the disclosure, the user authentication module 146 may compare a type of a motion identified based on the first signal and a biometric feature identified based on the second signal with the types of motions and the biometric features included in the authentication information, and acquire a score according to the comparison result. Then, if the acquired score is greater than or equal to a predetermined threshold value, the user authentication module 146 may identify that the user is a registered user, and output a result value indicating success of authentication. In contrast, if the acquired score is smaller than the predetermined threshold value, the user authentication module 146 may identify that the user is an unregistered user, and output a result value indicating failure of authentication.

If it is identified that the user is a registered user (i.e., user authentication succeeded) as a result that user authentication was performed, the processor 140 may release locking of the electronic device 100. When locking of the electronic device 100 is released, the processor 140 may allow the user to access information, such as a schedule, text messages, a contact list, or the like, stored in the electronic device 100, or use applications, such as a phone application, a payment application, a finance application, or the like. It is obvious that applications that can be accessed in case locking of the electronic device 100 is released and functions of the electronic device 100 can be changed according to the setting of the developer or the user.

If it is identified that the user is an unregistered user (i.e., user authentication failed) as a result that user authentication was performed, the processor 140 may maintain locking of the electronic device 100, and request additional authentication to the user. Also, if user authentication continuously fails by greater than or equal to a threshold number of times, the processor 140 may delete at least some data stored in the electronic device 100, or initialize the electronic device 100.

Meanwhile, if a score according to user authentication is smaller than a first threshold value which becomes a standard of success of authentication, and is greater than or equal to a second threshold value which becomes a standard of failure of authentication, the processor 140 may request reauthentication to the user. For example, the processor 140 may provide a message and/or a user interface for requesting reauthentication, such as authentication through an external device connected with the electronic device 100, authentication through a contact list, such as an e-mail of the user, or the like, authentication using other biometric information that was not used in an authentication process, or the like.

Meanwhile, as illustrated in FIG. 2, when user authentication is completed through the user authentication module 146, the processor 140 may update the authentication information through the authentication information update module 147. ‘The authentication information update module 147’ refers to a module that can update the authentication information by using the motion information and the biometric information used for user authentication.

According to one or more embodiments of the disclosure, if it is identified that the user is a registered user as a result that user authentication was performed, the processor 140 may update the authentication information based on the motion information and the biometric information. In other words, if it is identified that the motion information and the biometric information used for user authentication are information that can be trusted, the processor 140 may update (or calibrate) the authentication information by using the motion information and the biometric information used for user authentication.

In contrast, if it is identified that the user is an unregistered user as a result that user authentication was performed, the processor 140 may maintain locking of the electronic device 100, and may not update the authentication information by using the motion information and the biometric information used for user authentication.

According to the various embodiments that were described above with reference to FIGS. 1 and 2, in case a motion for user authentication is performed, the electronic device 100 may perform user authentication based on a biometric feature of the user according to the motion together with the type of the motion.

Also, as the electronic device 100 performs user authentication by combining the type of the user's motion and a biometric feature of the user, accuracy of user authentication can be improved. For example, the electronic device 100 may combine features of a bone conduction signal, a bioimpedance signal, and a heartbeat signal, or the like, which have high uniqueness for each user with a motion of the user that is performed in real time, and may thereby perform multi-modal user authentication which has high accuracy and convenience.

In addition, as the electronic device 100 does not need a special sensor, such as a fingerprint recognition sensor, a high performance camera, or the like, and collects various kinds of information through a sensor after a motion of the user starts, the electronic device 100 can perform user authentication effectively without big power consumption.

Further, as the electronic device 100 performs user authentication by identifying a motion of the user and whether the user biologically exists (i.e., liveness checking), high security can be secured. In particular, in case the electronic device 100 is implemented as a wearable device (e.g., a smart watch, or the like), the electronic device 100 can be utilized easily as an authentication means for another device of the user (e.g., a laptop, a vehicle, a digital door lock, or the like) or various services that require user authentication (e.g., a payment service, a financial service, or the like).

Further, if it is identified that the motion information and the biometric information used for user authentication are information that can be trusted, the electronic device 100 can further improve the reliability of user authentication whenever the number of times of user authentication increases by updating the authentication information by using the motion information and the biometric information used for user authentication.

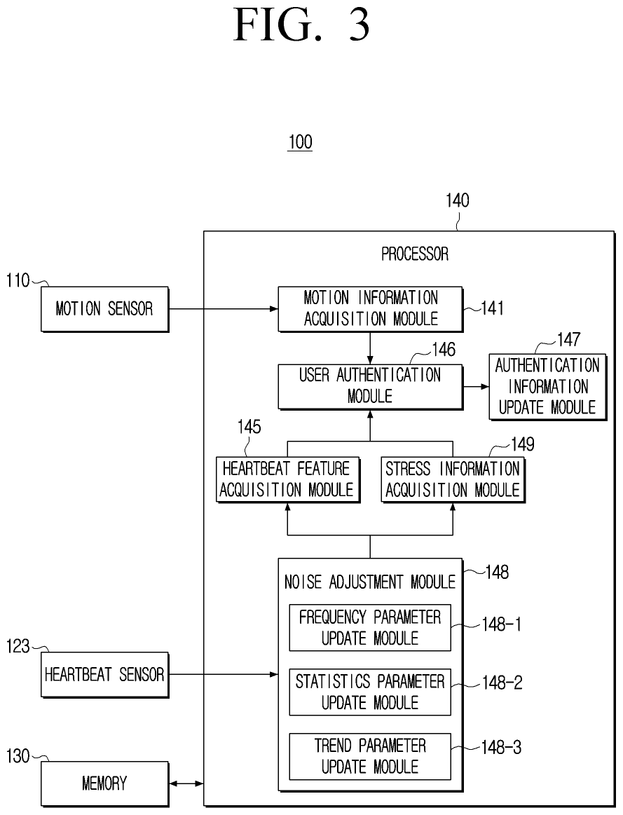

FIG. 3 is a block diagram illustrating a plurality of modules according to an embodiment of the disclosure.

Referring to FIG. 3, a plurality of modules according to the disclosure may include a noise adjustment module 148, a motion information acquisition module 141, a heartbeat feature acquisition module 145, a stress information acquisition module 149, a user authentication module 146, and an authentication information update module 147. Also, the processor 140 may perform various processing processes related to user authentication by using the plurality of modules as illustrated in FIG. 3. The plurality of modules may consist of hardware modules or software modules, and at least one module among the plurality of modules may be implemented through a neural network.

As described above, if the first signal is acquired through the motion sensor 110, the processor 140 may acquire motion information indicating the type of the user's motion by inputting the first signal into the motion information acquisition module 141. Specifically, the motion information acquisition module 141 may identify which type among the plurality of predefined motion types the user's motion corresponds to based on the first signal received through the at least one motion sensor 110.

In particular, in one or more embodiments wherein user authentication is performed by using a heartbeat signal, the term ‘a motion’ may be used as meaning including ‘an activity’ which means a collection of a series of motions. For example, a motion of the user may include activities, such as ‘walking,’ ‘running,’ ‘seating,’ and ‘sleeping,’ or the like. For example, the motion information acquisition module 141 may identify which activity among activities, such as ‘walking,’ ‘running,’ ‘seating,’ and ‘sleeping,’ or the like, a motion of the user corresponds to based on the first signal.

Meanwhile, if a heartbeat signal is acquired through the heartbeat sensor 123 included in the biometric sensor 120, the processor 140 may input the acquired heartbeat signal into the noise adjustment module 148, and adjust a noise included in the heartbeat signal.

The noise adjustment module 148 refers to a module that can adjust a noise included in a heartbeat signal. Specifically, if a request for adjusting a noise is received, the noise adjustment module 148 may update at least one parameter related to a noise of a heartbeat signal, for making a heartbeat feature extracted correctly. The noise adjustment module 148 may include at least one of a frequency parameter update module 148-1, a statistics parameter update module 148-2, or a trend parameter update module 148-3. In the description of the disclosure, ‘adjusting’ a noise may include ‘removing’ and ‘changing’ a noise of a heartbeat signal.

The frequency parameter update module 148-1 refers to a module that can update a frequency parameter related to a noise of a heartbeat signal. As an example, operations performed according to the frequency parameter update module 148-1 may be performed in an order as below.

The frequency parameter update module 148-1 may stabilize a heartbeat signal by removing a high frequency component of the heartbeat signal. In other words, the frequency parameter update module 148-1 may minimize irregular fluctuation and emphasize a main feature of a heartbeat signal by removing the trend of the signal by removing a high frequency component indicating a swift change of the heartbeat signal.

The frequency parameter update module 148-1 may perform a short-time fourier transformation for the heartbeat signal from which a high frequency component was removed, and may thereby express the heartbeat signal expressed as time series data in a form of a spectrum that can be analyzed in a frequency domain.

The frequency parameter update module 148-1 may remove the remaining frequency components excluding the band of interest in the heartbeat signal expressed in a form of a spectrum by performing bandpass filtering.

After bandpass filtering is performed, the frequency parameter update module 148-1 may measure power spectral density (PSD) indicating energy distribution of signals for each sub band. Then, the frequency parameter update module 148-1 may adjust a noise of the heartbeat signal by updating the frequency parameter based on the power spectral density.

The statistics parameter update module 148-2 refers to a module that can update a statistical parameter related to a noise of a heartbeat signal. As an example, operations performed according to the statistics parameter update module 148-2 may be performed in an order as below.

The statistics parameter update module 148-2 may stabilize a heartbeat signal by removing a low frequency component of the heartbeat signal. In other words, the statistics parameter update module 148-2 may remove an impulse of the signal by removing a low frequency component indicating a slow change of the heartbeat signal, and may accordingly make the heartbeat signal soft and reduce noises.

The statistics parameter update module 148-2 may apply an autoregressive moving average model (ARMA) for the heartbeat signal from which a low frequency component was removed. The ARMA model is a type of statistics model that is used for analyzing and predicting time series data, and refers to a technic for explaining a feature of time series data by combining an autoregressive parameter and a moving average parameter.

Specifically, the statistics parameter update module 148-2 may assume an autoregressive parameter indicating a relation between the data of the current time and the data of the previous time by using a technic, such as an autoregressive parameter least square method. Also, the statistics parameter update module 148-2 may assume a moving average parameter indicating a relation between the data of the current time and the data of the previous time by using a technic, such as a least square method or a maximum likelihood method. The statistics parameter update module 148-2 may adjust a noise of the heartbeat signal by updating the autoregressive parameter and the moving average parameter.

The trend parameter update module 148-3 refers to a module that can update a trend parameter related to a noise of a heartbeat signal. Here, the trend parameter refers to a parameter that indicates what a trend, i.e., a tendency of a heartbeat signal is like, and it may be replaced by a term, such as a tendency parameter. As an example, operations performed according to the trend parameter update module 148-3 may be performed in an order as below.

The trend parameter update module 148-3 may decompose a heartbeat signal into trend components in an N number and residuals by using an empirical mode decomposition (EMD) technic. Here, the EMD refers to a technic of decomposing a heartbeat signal expressed as time series data into trend components in various frequency bands.

The trend parameter update module 148-3 may separate the trend components from the heartbeat signal by removing the trend components in the N number assumed through the EMD technic from the heartbeat signal.

The trend parameter update module 148-3 may identify whether the residuals which are the remaining parts when the trend components were separated from the heartbeat signal are similar to a white noise. Specifically, the trend parameter update module 148-3 may identify whether the residuals are similar to a white noise by using a standard statistical certification technic, such as Kolmogorov-Smirnov certification.

If the residuals are not similar to a white noise, the trend parameter update module 148-3 may increase the mode number N and apply the N-level EMD again, and decompose the heartbeat signal into trend components in an N+1 number. For example, if the residuals are not similar to a white noise, the trend parameter update module 148-3 may perform decomposition of the trend components repeatedly to a desired level.

In contrast, if the residuals are similar to a white noise, the trend parameter update module 148-3 may assume an intrinsic mode function (IMF) which is a function indicating a specific trend corresponding to a specific frequency band of the signal, and model the statistical feature of the residuals and update the trend parameter based on the assumed IMF, and may thereby adjust the noise of the heartbeat signal.

If the noise included in the heartbeat signal is adjusted as described above, the processor 140 may input the heartbeat signal of which noise was adjusted into the heartbeat feature acquisition module 145 and the stress information acquisition module 149, and acquire information on the heartbeat feature and stress information. Meanwhile, depending on embodiments of the disclosure, the processor 140 may input a heartbeat signal into the heartbeat feature acquisition module 145 and the stress information acquisition module 149 without performing a noise adjustment process for the heartbeat signal, and acquire information on the heartbeat feature and stress information.

The heartbeat feature acquisition module 145 refers to a module that can acquire information on a heartbeat feature transmitted from the user's heart while a motion of the user is being performed. As the process of acquiring information on a heartbeat feature was described with reference to FIGS. 1 and 2, overlapping explanation regarding the same content will be omitted.

The stress information acquisition module 149 refers to a module that can acquire stress information based on a heartbeat signal. Here, ‘stress information’ generally refers to information indicating which level the stress level of the user is. In particular, stress information may indicate a stress level of the user according to a motion of the user. As an example, operations performed according to the stress information acquisition module 149 may be performed in an order as below.

The stress information acquisition module 149 may perform pre-processing for removing at least one of noises and/or trends included in a heartbeat signal. As the pre-processing process for removing noises and trends was described above in the explanation regarding the pre-processing process of the first signal and the second signal, overlapping explanation regarding the same content will be omitted.

After performing the pre-processing process, the stress information acquisition module 149 may detect R-peaks by using an electrocardiogram processing algorithm, such as a Pan-Tompkins algorithm. Here, the R-peaks are signals indicating a main event of the heartbeat in the heartbeat signal, and may be a reference point for the heartbeat signal.

The stress information acquisition module 149 may assume variability of the heartbeat by calculating intervals among the R-peaks (i.e., R-to-R durations). For example, the stress information acquisition module 149 may assume heart rate variability (HRV) indicating variability of the heartbeat intervals according to the posture, the activity, and the emotion, or the like, of the user.

The stress information acquisition module 149 may identify a stress level of the user corresponding to the heartbeat signal based on the assumed variability of the heartbeat. Specifically, the stress information acquisition module 149 may acquire stress information indicating a stress level of the user by using a regression model predicting a stress level.

When the motion information, the information on the heartbeat feature, and the stress information are acquired as described above, the processor 140 may input the motion information, the information on the heartbeat feature, and the stress information into the user authentication module 146, and perform user authentication according to whether the type of the motion, the heartbeat feature, and the stress level match the authentication information.

The user authentication module 146 refers to a module that performs user authentication as described above, and may include a user authentication module including a neural network. Meanwhile, in the explanation regarding FIGS. 1 and 2, embodiments wherein the user authentication module 146 performs user authentication based on the motion information and the biometric information were explained, but the user authentication module 146 may perform user authentication by using the motion information, the information on the heartbeat feature, and the stress information.

The user authentication module 146 may compare the type of the motion identified based on the first signal, the stress level identified based on the second signal (specifically, the heartbeat signal), and the heartbeat feature identified based on the heartbeat signal with the types of motions, the stress levels, and the heartbeat features included in the authentication information, and acquire a score according to the comparison result. Then, if the acquired store is greater than or equal to a predetermined threshold value, the user authentication module 146 may identify that the user is a registered user, and output a result value indicating success of authentication. In contrast, if the acquired store is smaller than the predetermined threshold value, the user authentication module 146 may identify that the user is an unregistered user, and output a result value indicating failure of authentication.

A matching process between information performed in the user authentication module 146 may be performed by converting each of the information into a vector, and based on whether the features indicated by each vector are similar. Also, the user authentication module 146 may perform the matching process by acquiring the first vector corresponding to the type of the motion identified based on the first signal, the second vector corresponding to the stress level identified based on the second signal, and the third vector corresponding to the heartbeat feature identified based on the second signal, and acquiring the fourth vector by combining the first vector, the second vector, and the third vector, and comparing the fourth vector with the vectors stored in the authentication information.

According to one or more embodiments of the disclosure, the processor 140 may use a dynamic time warping (DTW) technic in a process of comparing a heartbeat signal received through the heartbeat sensor 123 and a heartbeat signal included in the authentication information. Here, the dynamic time warping technic refers to a technic of compensating a temporal difference between two time series data, and aligning similar heartbeat patterns and comparing them. Specifically, the dynamic time warping technic may include a process of calculating a distance matrix between two heartbeat signals to be compared, and calculating an optimal alignment route based on the distance matrix, and aligning the two heartbeat signals according to the calculated optimal alignment route.

Meanwhile, as illustrated in FIG. 3, when user authentication is completed through the user authentication module 146, the processor 140 may update the authentication information through the authentication information update module 147. The authentication information update module 147 refers to a module that can update the authentication information by using the motion information and the biometric information used for user authentication as described above.

If it is identified that the user is a registered user as a result that user authentication was performed, the processor 140 may update the authentication information based on the motion information, the information on the heartbeat feature, and the stress information. In other words, if it is identified that the motion information, the information on the heartbeat feature, and the stress information used for user authentication are information that can be trusted, the processor 140 may update the authentication information by using the motion information, the information on the heartbeat feature, and the stress information used for user authentication.

In contrast, if it is identified that the user is an unregistered user as a result that user authentication was performed, the processor 140 may maintain locking of the electronic device 100, and may not update the authentication information by using the motion information, the information on the heartbeat feature, and the stress information used for user authentication.

Meanwhile, update of the authentication information through the authentication information update module 147 may be repeatedly performed whenever user authentication is performed. Also, collection of signals through the motion sensor 110 and the biometric sensor 120 may be repeated per predetermined period. Here, the feature of collecting signals per predetermined period may mean that signals may be collected when user authentication is not performed. In particular, if the authentication information is updated by periodically acquiring a heartbeat signal, the authentication information can be personalized to fit the unique biometric feature of the individual user effectively.

Meanwhile, in the explanation regarding FIG. 3, an embodiment wherein motion information is acquired based on the first signal acquired through the motion sensor 110 was explained, but the disclosure is not limited thereto. For example, the processor 140 may identify the type of the user's motion by using not only the first signal but also the information on the heartbeat feature and the stress information together. For example, the processor 140 may identify that a motion of the user corresponds to ‘sleeping’ based on whether the stress level of the user is smaller than a threshold level, together with whether the first signal and the heartbeat feature are similar to the feature indicating the activity which is ‘sleeping.’

Meanwhile, in FIG. 3, only the heartbeat sensor 123 among the biometric sensors 120 was illustrated, but a bone conduction signal and a bioimpedance signal acquired through the bone conduction sensor 121 and the bioimpedance sensor 122 may be used in a process of identifying the type of the user's motion, or may be used in a process of identifying a stress level of the user.

According to the various embodiments described above with reference to FIG. 3, the electronic device 100 may perform user authentication by combining a heartbeat signal having high uniqueness for the user with a motion of the user having a high real-time property, and may thereby perform user authentication which is convenient and transparent, and also has high accuracy in various external conditions, such as cases wherein the user is exercising or the lighting is dark.

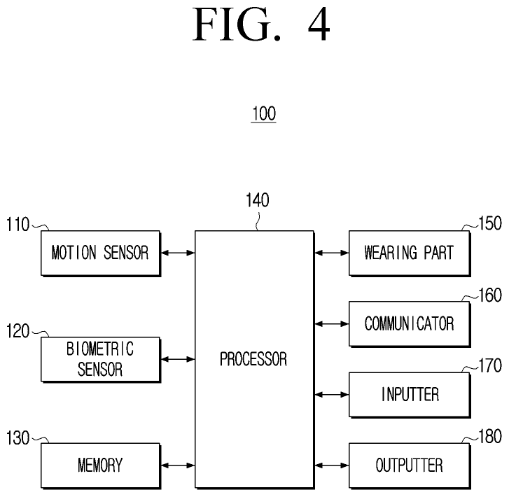

FIG. 4 is a block diagram illustrating a configuration of an electronic device according to an embodiment of the disclosure.

Referring to FIG. 4, the electronic device 100 may include not only the motion sensor 110, the biometric sensor 120, the memory 130, and the processor 140, but may also further include a wearing part 150, a communicator 160, an inputter 170, and an outputter 180. However, in carrying out the disclosure, new components can be added in addition to the components as illustrated in FIGS. 1 and 4, or some components can be omitted.

The wearing part 150 refers to a component that enables the user to wear the electronic device 100. For example, in case the electronic device 100 is implemented as a smart watch, the wearing part 150 may include a watch strap (a strap or a band) and a buckle, or the like, for fixing the smart watch on the wrist of the user.

In case the electronic device 100 is implemented as smart glasses, the wearing part 150 may include legs of the glasses (temples) and nose pads, or the like. Other than the above, a component among the components of the electronic device 100 that can contact at least some of the body parts of the user in case the user wears the electronic device 100, such as the rear surface of the smart watch, or the like, may correspond to the wearing part 150 according to the disclosure.

According to one or more embodiments of the disclosure, at least one sensor included in the at least one motion sensor 110 and the at least one biometric sensor 120 may be coupled to the wearing part 150. For example, in case the electronic device 100 is implemented as a smart watch, the bone conduction sensor 121, the bioimpedance sensor 122, the heartbeat sensor 123, or the like, may be coupled to the rear surface of the watch and/or the watch strap so that they can contact the user's skin. For example, the wearing part 150 may be a smart strap developed for extending the functions of the smart watch or providing additional functions.

The communicator 160 may include a circuit, and perform communication with an external device. Specifically, the processor 140 may receive various kinds of data or information from an external device connected through the communicator 160, or transmit various kinds of data or information to the external device.

The communicator 160 may include at least one of a Wi-Fi module, a Bluetooth module, a wireless communication module, an NFC module, or an ultra wide band (UWB) module. Specifically, each of the Wi-Fi module and the Bluetooth module may perform communication by a Wi-Fi method and a Bluetooth method. In the case of using a Wi-Fi module or a Bluetooth module, various types of connection information, such as a service set identifier (SSID), or the like, is transmitted and received first, and connection of communication is performed by using the information, and various types of information can be transmitted and received thereafter.

Also, a wireless communication module may perform communication according to various communication standards, such as institute of electrical and electronics engineers (IEEE), Zigbee, 3^{rd} generation (3G), 3^{rd} generation partnership project (3GPP), long term evolution (LTE), and 5^{th} generation (5G), or the like. In addition, an NFC module may perform communication by a near field communication (NFC) method of using a 13.56 MHz band among various radio frequency identification (RF-ID) frequency bands, such as 135 kHz, 13.56 MHz, 433 MHZ, 860-960 MHz, and 2.45 GHZ, or the like. Further, a UWB module can correctly measure a time of arrival (ToA) which is the time that a pulse reaches a target, and an angle of arrival (AoA) which is a pulse arrival angle in a transmission device through communication between UWB antennas, and accordingly, the UWB module can perform precise distance and location recognition in an error range of within scores of cm indoors.

According to one or more embodiments of the disclosure, if it is identified that the user is an unregistered user as a result that user authentication was performed, the processor 140 may maintain locking of the electronic device 100, and control the communicator 160 to transmit a message for guiding that authentication by an unregistered user was performed to an external device connected to the electronic device 100.

The inputter 170 may include a circuit, and the processor 140 may receive a user instruction for controlling the operations of the electronic device 100 through the inputter 170. Specifically, the inputter 170 may consist of components, such as a microphone, a camera, and a remote control signal receiver, or the like. Also, the inputter 170 may be implemented in a form of being included in a display as a touch screen. In particular, the microphone may receive a voice signal, and convert the received voice signal into an electric signal.

According to one or more embodiments of the disclosure, the processor 140 may receive a user input, such as a user input for initiating user authentication, a user input for user registration, or the like, through the inputter 170.

The outputter 180 may include a circuit, and the processor 140 may output various functions that the electronic device 100 can perform through the outputter 180. Also, the outputter 180 may include at least one of a display, a speaker, or an indicator.

The display may output image data by control by the processor 140. Specifically, the display may output an image stored in advance in the memory 130 by control by the processor 140. In particular, the display according to an embodiment of the disclosure may display a user interface stored in the memory 130. The display may be implemented as a liquid crystal display (LCD) panel, organic light emitting diodes (OLED), or the like, and the display may also be implemented as a flexible display, a transparent display, or the like, depending on cases. However, the display according to the disclosure is not limited to specific types.

The speaker may output audio data by control by the processor 140, and the indicator may be lighted by control by the processor 140.

According to one or more embodiments of the disclosure, if it is identified that the user is an unregistered user as a result that user authentication was performed, the processor 140 may maintain locking of the electronic device 100, and control the outputter 180 to output a guide message for requesting additional authentication.

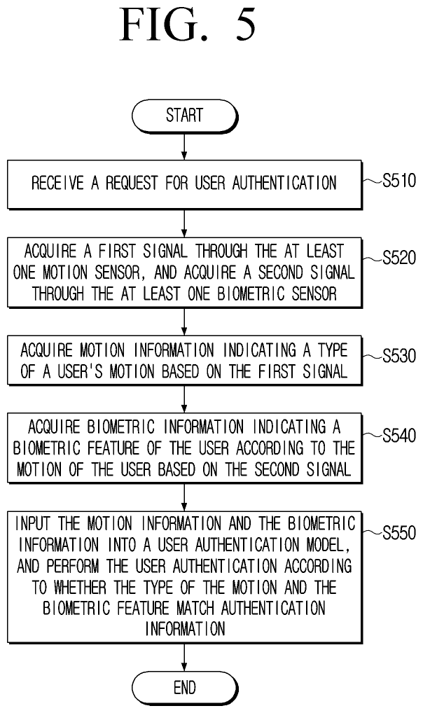

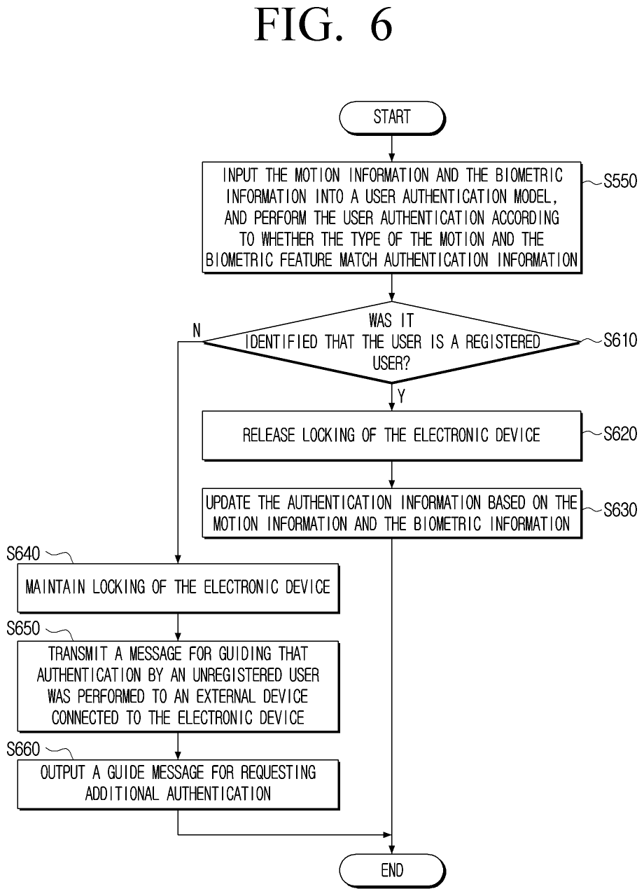

FIGS. 5 and 6 are flowcharts illustrating a controlling method of the electronic device 100 according to various embodiments of the disclosure.

Referring to FIG. 5, the electronic device 100 may receive a request for user authentication in operation S510. When a request for user authentication is received, the electronic device 100 may acquire the first signal through the at least one motion sensor 110, and acquire the second signal through the at least one biometric sensor 120 in operation S520.

According to one or more embodiments of the disclosure, a request for user authentication may be received based on a user input for initiating user authentication, or received based on generation of an event which was defined in advance that user authentication is necessary.

According to one or more embodiments of the disclosure, when the first signal is received through the at least one motion sensor 110, the electronic device 100 may acquire the second signal through the at least one biometric sensor 120. For example, the electronic device 100 may acquire the second signal through the at least one biometric sensor 120 during a predetermined period after the first signal started to be received through the at least one motion sensor 110 according to a motion of the user.

When the first signal is acquired, the electronic device 100 may acquire motion information indicating the type of the user's motion based on the first signal in operation S530. Acquiring motion information indicating the type of the user's motion may include a process of identifying which type among the plurality of predefined motion types the user's motion indicated by the feature of the first signal corresponds to.

When the second signal is acquired, the electronic device 100 may acquire biometric information indicating a biometric feature of the user according to the motion of the user based on the second signal in operation S540. The at least one biometric sensor 120 may include at least one of the bone conduction sensor 121, the bioimpedance sensor 122, or the heartbeat sensor 123, and the electronic device 100 may receive the second signal through each of the bone conduction sensor 121, the bioimpedance sensor 122, and the heartbeat sensor 123. Also, the electronic device 100 may acquire information on an audio feature transmitted from at least of the bone or the joint according to a motion of the user, information on an electrical feature transmitted from at least one of the muscle, the tissue, or the joint of the user according to a motion of the user, and information on a heartbeat feature transmitted from the heart of the user while a motion of the user is being performed based on the second signal.

The electronic device 100 may input the motion information and the biometric information into the user authentication model, and perform user authentication according to whether the type of the motion and the biometric feature match the authentication information in operation S550.

‘The user authentication model’ refers to a neural network model trained to perform user authentication. The user authentication model may be trained to identify whether input motion information and biometric information match authentication information that was constructed in advance. Specifically, the electronic device 100 may acquire a third vector by combining the first vector corresponding to the motion information and the second vector corresponding to the biometric information, and input the acquired third vector into the user authentication model. When the third vector is input, the user authentication model may identify whether the feature indicated by the third vector matches the features of the authentication information constructed in advance, and output information regarding the identification result.

According to one or more embodiments of the disclosure, the electronic device 100 may compare the type of the motion identified based on the first signal and the biometric feature identified based on the second signal with the types of motions and the biometric features included in the authentication information, and acquire a score according to the comparison result. Then, if the acquired score is greater than or equal to a predetermined threshold value, the electronic device 100 may identify that the user is a registered user, and output a result value indicating success of authentication. In contrast, if the acquired score is smaller than the predetermined threshold value, the electronic device 100 may identify that the user is an unregistered user, and output a result value indicating failure of authentication.

FIG. 6 is a flowchart for illustrating an operation of the electronic device 100 according to a result that user authentication was performed according to the operation S550 in FIG. 5.

Referring to FIG. 6, if it is identified that the user is a registered user as a result that user authentication was performed in operation S610-Y, the electronic device 100 may release locking of the electronic device 100 in operation S620. When locking of the electronic device 100 is released, the electronic device 100 may allow the user to access information, such as a schedule, text messages, a contact list, or the like, stored in the electronic device 100, or use applications, such as a phone application, a payment application, a finance application, or the like.

Also, if it is identified that the user is a registered user as a result that user authentication was performed in operation S610-Y, the electronic device 100 may update the authentication information based on the motion information and the biometric information in operation S630. In other words, if it is identified that the motion information and the biometric information used for user authentication are information that can be trusted, the electronic device 100 may update (or calibrate) the authentication information by using the motion information and the biometric information used for user authentication.

If it is identified that the user is an unregistered user as a result that user authentication was performed in operation S610-N, the electronic device 100 may maintain locking of the electronic device 100 in operation S640. Also, if it is identified that the user is an unregistered user as a result that user authentication was performed in operation S610-N, the electronic device 100 may transmit a message for guiding that authentication by an unregistered user was performed to an external device connected to the electronic device 100 in operation S650, and output a guide message for requesting additional authentication in operation S660. Also, if it is identified that the user is an unregistered user as a result that user authentication was performed in operation S610-N, the electronic device 100 may not update the authentication information by using the motion information and the biometric information used for user authentication.

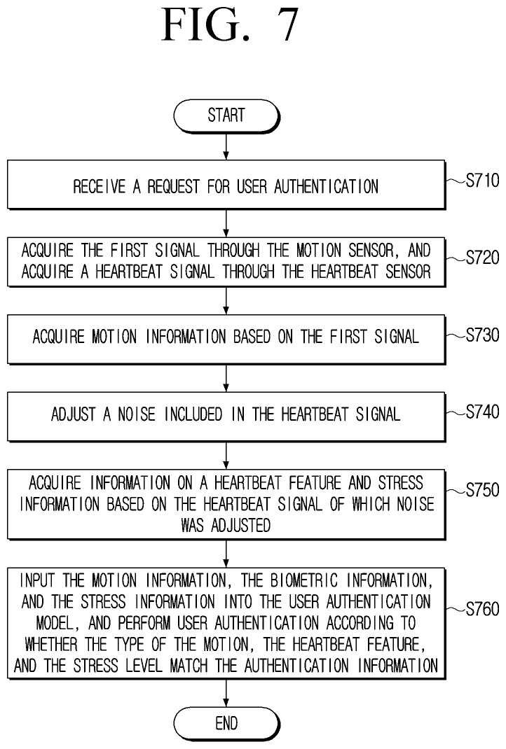

FIG. 7 is a flowchart illustrating a controlling method of the electronic device 100 according to an embodiment of the disclosure.

Referring to FIG. 7, the electronic device 100 may receive a request for user authentication in operation S710. When a request for user authentication is received, the electronic device 100 may acquire the first signal through the motion sensor 110, and acquire a heartbeat signal through the heartbeat sensor 123 in operation S720.

When the first signal is acquired through the motion sensor 110, the electronic device 100 may acquire motion information based on the first signal in operation S730. Specifically, the electronic device 100 may identify which type among the plurality of predefined motion types the user's motion corresponds to based on the first signal received through the at least one motion sensor 110. For example, the electronic device 100 may identify which activity among activities, such as ‘walking,’ ‘running,’ ‘seating,’ and ‘sleeping,’ or the like, a motion of the user corresponds to based on the first signal.

When a heartbeat signal is acquired through the heartbeat sensor 123, the electronic device 100 may adjust a noise included in the heartbeat signal in operation S740. Specifically, the electronic device 100 may stabilize the heartbeat signal by adjusting at least one of a frequency parameter, a statistics parameter, or a trend parameter related to the heartbeat signal.

The electronic device 100 may acquire information on a heartbeat feature and stress information based on the heartbeat signal of which noise was adjusted in operation S750. Specifically, the electronic device 100 may acquire information on a heartbeat feature based on the heartbeat signal of which noise was adjusted, and may also acquire stress information indicating the stress level of the user based on the heartbeat signal of which noise was adjusted. The electronic device 100 may input the motion information, the biometric information, and the stress information into the user authentication model, and perform user authentication according to whether the type of the motion, the heartbeat feature, and the stress level match the authentication information in operation S760.

Specifically, the electronic device 100 may compare the type of the motion identified based on the first signal, the stress level identified based on the heartbeat signal, and the heartbeat feature identified based on the heartbeat signal with the type of the motion, the stress level, and the heartbeat feature included in the authentication information, and acquire a score according to the comparison result. Then, if the acquired score is greater than or equal to the predetermined threshold value, the electronic device 100 may identify that the user is a registered user, and output a result value indicating success of authentication. In contrast, if the acquired score is smaller than the predetermined threshold value, the electronic device 100 may identify that the user is an unregistered user, and output a result value indicating failure of authentication.

Meanwhile, if it is identified that the user is a registered user as a result that user authentication was performed, the electronic device 100 may update the authentication information based on the motion information, the information on the heartbeat feature, and the stress information. In contrast, if it is identified that the user is an unregistered user as a result that user authentication was performed, the electronic device 100 may maintain locking of the electronic device 100, and may not update the authentication information by using the motion information, the information on the heartbeat feature, and the stress information used for user authentication.

Meanwhile, the controlling method of the electronic device 100 according to the aforementioned embodiments may be implemented as a program and provided to the electronic device 100. In particular, a program including the controlling method of the electronic device 100 may be provided while being stored in a non-transitory computer readable medium.

Specifically, in a non-transitory computer readable recording medium including a program executing a controlling method of the electronic device 100, the controlling method of the electronic device 100 may include, based on receiving a request for user authentication, acquiring a first signal through the at least one motion sensor 110, and acquiring a second signal through the at least one biometric sensor 120, acquiring motion information indicating a type of a user's motion based on the first signal, acquiring biometric information indicating a biometric feature of the user according to the motion of the user based on the second signal, and inputting the motion information and the biometric information into a user authentication model, and performing the user authentication according to whether the type of the motion and the biometric feature match authentication information.

While a controlling method of the electronic device 100, and a computer readable recording medium including a program executing the controlling method of the electronic device 100 were explained briefly above, this is only for omitting overlapping explanation, and the various embodiments regarding the electronic device 100 can obviously be applied to the controlling method of the electronic device 100, and the computer readable recording medium including a program executing the controlling method of the electronic device 100.

According to the aforementioned various embodiments of the disclosure, in case a motion for user authentication is performed, the electronic device 100 may perform user authentication based on a biometric feature of the user according to the motion together with the type of the motion.

Also, as the electronic device 100 performs user authentication by combining the type of the user's motion and the biometric feature of the user, accuracy of the user authentication can be improved. For example, the electronic device 100 may combine features of a bone conduction signal, a bioimpedance signal, and a heartbeat signal, or the like, having high uniqueness for each user with a motion of the user that is performed in real time, and may thereby perform multi-modal user authentication which has high accuracy and convenience.

In addition, as the electronic device 100 does not need a special sensor, such as a fingerprint recognition sensor, a high performance camera, or the like, and collects various kinds of information through a sensor after a motion of the user starts, the electronic device 100 can perform user authentication effectively without big power consumption.

Further, as the electronic device 100 performs user authentication by identifying a motion of the user and whether the user biologically exists (i.e., liveness checking), high security can be secured. In particular, in case the electronic device 100 is implemented as a wearable device (e.g., a smart watch, or the like), the electronic device 100 can be utilized easily as an authentication means for another device of the user (e.g., a laptop, a vehicle, a digital door lock, or the like) or various services that require user authentication (e.g., a payment service, a financial service, or the like).

Also, if it is identified that the motion information and the biometric information used for user authentication are information that can be trusted, the electronic device 100 can further improve the reliability of user authentication whenever the number of times of user authentication increases by updating the authentication information by using the motion information and the biometric information used for user authentication.

Further, the electronic device 100 may perform user authentication by combining a heartbeat signal having high uniqueness for the user with a motion of the user having a high real-time property, and may thereby perform user authentication which is convenient and transparent, and also has high accuracy in various external conditions, such as cases wherein the user is exercising or the lighting is dark.

Functions related to artificial intelligence according to the disclosure are operated through the processor 140 and the memory 130 of the electronic device 100.

The processor 140 may consist of one or a plurality of processors 140. Here, the one or plurality of processors 140 may include at least one of a central processing unit (CPU), a graphics processing unit (GPU), or a neural processing unit (NPU), but are not limited to the aforementioned examples of the processor 140.

A CPU is a generic-purpose processor 140 that can perform not only general operations but also artificial intelligence operations, and it can effectively execute a complex program through a multilayer cache structure. A CPU is advantageous for a serial processing method that enables a systemic linking between the previous calculation result and the next calculation result through sequential calculations. The generic-purpose processor 140 is not limited to the aforementioned examples excluding cases wherein it is specified as the aforementioned CPU.

A GPU is a processor 140 for mass operations, such as a floating point operation used for graphic processing, or the like, and it can perform mass operations in parallel by massively integrating cores. In particular, a GPU may be advantageous for a parallel processing method, such as a convolution operation, or the like, compared to a CPU. Also, a GPU may be used as a co-processor 140 for supplementing the function of a CPU. The processor 140 for mass operations is not limited to the aforementioned examples excluding cases wherein it is specified as the aforementioned GPU.

An NPU is a processor 140 specialized for an artificial intelligence operation using an artificial neural network, and it can implement each layer constituting an artificial neural network as hardware (e.g., silicon). Here, the NPU is designed to be specialized according to the required specification of a company, and thus it has a lower degree of freedom compared to a CPU or a GPU, but it can effectively process an artificial intelligence operation required by the company. Meanwhile, as the processor 140 specialized for an artificial intelligence operation, the NPU may be implemented in various forms, such as a tensor processing unit (TPU), an intelligence processing unit (IPU), a vision processing unit (VPU), or the like. Meanwhile, the artificial intelligence processor 140 is not limited to the aforementioned examples excluding cases wherein it is specified as the aforementioned NPU.

Also, the one or plurality of processors 140 may be implemented as a system on chip (SoC). Here, in the SoC, the memory 130, and a network interface, such as a bus for data communication between the processor 140 and the memory 130, or the like, may be further included other than the one or plurality of processors 140.

In case the plurality of processors 140 are included in the system on chip (SoC) included in the electronic device 100, the electronic device 100 may perform an operation related to artificial intelligence (e.g., an operation related to learning or inference of the artificial intelligence model) by using some processors 140 among the plurality of processors 140. For example, the electronic device 100 may perform an operation related to artificial intelligence by using at least one of a GPU, an NPU, a VPU, a TPU, or a hardware accelerator specified for artificial intelligence operations, such as a convolution operation, a matrix product operation, or the like, among the plurality of processors 140. However, this is merely an example, and the electronic device 100 can obviously process an operation related to artificial intelligence by using the generic-purpose processor 140, such as a CPU, or the like.

Also, the electronic device 100 may perform operations for functions related to artificial intelligence by using a multicore (e.g., a dual core, a quad core, or the like) included in one processor 140. More particularly, the electronic device 100 may perform artificial intelligence operations, such as a convolution operation, a matrix product operation, or the like, in parallel by using the multicore included in the processor 140.

The one or plurality of processors 140 perform control to process input data according to predefined operation rules or an artificial intelligence model stored in the memory 130. The predefined operation rules or the artificial intelligence model are characterized in that they are made through learning.

Here, being made through learning means that predefined operation rules or artificial intelligence models having desired characteristics are made by applying a learning algorithm to a plurality of learning data. Such learning may be performed in a device itself wherein artificial intelligence is performed according to the disclosure, or through a separate server/system.

An artificial intelligence model may consist of a plurality of neural network layers. At least one layer has at least one weight value, and performs an operation of the layer through the operation result of the previous layer and at least one defined operation. As examples of a neural network, there are a convolutional neural network (CNN), a deep neural network (DNN), a recurrent neural network (RNN), a restricted Boltzmann machine (RBM), a deep belief network (DBN), a bidirectional recurrent deep neural network (BRDNN), deep Q-networks, and a Transformer, but the neural network in the disclosure is not limited to the aforementioned examples excluding specified cases.

A learning algorithm is a method of training a specific subject device (e.g., a robot) by using a plurality of learning data and thereby making the specific subject device make a decision or make prediction by itself. As examples of learning algorithms, there are supervised learning, unsupervised learning, semi-supervised learning, or reinforcement learning, but learning algorithms in the disclosure are not limited to the aforementioned examples excluding specified cases.

A storage medium that is readable by machines may be provided in the form of a non-transitory storage medium. Here, the term ‘a non-transitory storage medium’ only means that the device is a tangible device, and does not include a signal (e.g., an electronic wave), and the term does not distinguish a case wherein data is stored semi-permanently in a storage medium and a case wherein data is stored temporarily. For example, ‘a non-transitory storage medium’ may include a buffer wherein data is temporarily stored.

According to an embodiment of the disclosure, methods according to the various embodiments disclosed herein may be provided while being included in a computer program product. A computer program product refers to a product, and it can be traded between a seller and a buyer. A computer program product can be distributed in the form of a storage medium that is readable by machines (e.g., compact disc read only memory (CD-ROM)), or distributed directly on-line (e.g., download or upload) through an application store (e.g., Play Store™), or between two user devices (e.g., smartphones). In the case of on-line distribution, at least a portion of a computer program product (e.g., a downloadable app) may be stored in a storage medium readable by machines, such as the server of the manufacturer, the server of the application store, and the memory 130 of the relay server at least temporarily, or may be generated temporarily.

In addition, each of the components (e.g., a module or a program) according to the aforementioned various embodiments of the disclosure may consist of a singular object or a plurality of objects. In addition, among the aforementioned corresponding sub components, some sub components may be omitted, or other sub components may be further included in the various embodiments. Alternatively or additionally, some components (e.g., a module or a program) may be integrated as an object, and perform functions that were performed by each of the components before integration identically or in a similar manner.

Further, operations performed by a module, a program, or other components according to the various embodiments may be executed sequentially, in parallel, repetitively, or heuristically. Alternatively, at least some of the operations may be executed in a different order or omitted, or other operations may be added.

Meanwhile, the term “a part” or “a module” used in the disclosure may include a unit consisting of hardware, software, or firmware, and may be interchangeably used with, for example, terms, such as a logic, a logical block, a component, or a circuit. In addition, “a part” or “a module” may be a component constituted as an integrated body or a minimum unit or a part thereof performing one or more functions. For example, a module may be constituted as an application-specific integrated circuit (ASIC).

Also, the various embodiments of the disclosure may be implemented as software including instructions stored in machine-readable storage media, which can be read by machines (e.g., computers). The machines refer to devices that call instructions stored in a storage medium, and can operate according to the called instructions, and the devices may include an electronic device according to the aforementioned embodiments (e.g., the electronic device 100).

In case an instruction is executed by a processor, the processor may perform a function corresponding to the instruction by itself, or by using other components under its control. An instruction may include a code that is generated or executed by a compiler or an interpreter.

It will be appreciated that various embodiments of the disclosure according to the claims and description in the specification can be realized in the form of hardware, software or a combination of hardware and software.

Any such software may be stored in non-transitory computer readable storage media. The non-transitory computer readable storage media store one or more computer programs (software modules), the one or more computer programs include computer-executable instructions that, when executed by one or more processors of an electronic device, cause the electronic device to perform a method of the disclosure.

Any such software may be stored in the form of volatile or non-volatile storage such as, for example, a storage device like read only memory (ROM), whether erasable or rewritable or not, or in the form of memory such as, for example, random access memory (RAM), memory chips, device or integrated circuits or on an optically or magnetically readable medium such as, for example, a compact disk (CD), digital versatile disc (DVD), magnetic disk or magnetic tape or the like. It will be appreciated that the storage devices and storage media are various embodiments of non-transitory machine-readable storage that are suitable for storing a computer program or computer programs comprising instructions that, when executed, implement various embodiments of the disclosure. Accordingly, various embodiments provide a program comprising code for implementing apparatus or a method as claimed in any one of the claims of this specification and a non-transitory machine-readable storage storing such a program.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. An electronic device comprising:
at least one motion sensor;
at least one biometric sensor;
memory storing one or more computer programs; and
one or more processors communicatively coupled to the at least one motion sensor, the at least one biometric sensor, and the memory,
wherein the one or more computer programs include computer-executable instructions that, when executed by the one or more processors, cause the electronic device to:
based on receiving a request for user authentication, acquire a first signal through the at least one motion sensor, and acquire a second signal through the at least one biometric sensor,
acquire motion information indicating a type of a user's motion based on the first signal,
acquire biometric information indicating a biometric feature of a user according to the motion of the user based on the second signal, and
input the motion information and the biometric information into a user authentication model, and perform the user authentication according to whether the type of the motion and the biometric feature match authentication information.

2. The electronic device of claim 1, wherein the one or more computer programs further include computer-executable instructions that, when executed by the one or more processors, cause the electronic device to:
based on identifying that the user is a registered user as a result that the user authentication was performed, release locking of the electronic device, and
update the authentication information based on the motion information and the biometric information.

3. The electronic device of claim 1, wherein the one or more computer programs further include computer-executable instructions that, when executed by the one or more processors, cause the electronic device to:
based on the first signal starting to be received through the at least one motion sensor, acquire a second signal through the at least one biometric sensor.

4. The electronic device of claim 1,
wherein the at least one biometric sensor comprises a bone conduction sensor, and
wherein the biometric information comprises information on an audio feature transmitted from at least one of a bone or a joint of the user according to the motion of the user.

5. The electronic device of claim 1,
wherein the at least one biometric sensor comprises a bioimpedance sensor, and
wherein the biometric information comprises information on an electrical feature transmitted from at least one of a muscle, a tissue, or a joint of the user according to the motion of the user.

6. The electronic device of claim 1,
wherein the at least one biometric sensor comprises a heartbeat sensor, and
wherein the biometric information comprises information on a heart rate feature transmitted from a heart of the user while the motion of the user is being performed.

7. The electronic device of claim 6,
wherein the one or more computer programs further include computer-executable instructions that, when executed by the one or more processors, cause the electronic device to:
acquire stress information indicating a stress level of the user based on the biometric information, and
wherein the user authentication model performs the user authentication according to whether the stress level, the type of the motion, and the heart rate feature match the authentication information.

8. The electronic device of claim 1, wherein the one or more computer programs further include computer-executable instructions that, when executed by the one or more processors, cause the electronic device to:
perform pre-processing for removing at least one of noises or trends of the first signal and the second signal; and
acquire the motion information and the biometric information based on the pre-processed first signal and the pre-processed second signal.

9. The electronic device of claim 1, further comprising:
a wearing part,
wherein one or more sensors included in the at least one motion sensor and the at least one biometric sensor are coupled to the wearing part.

10. The electronic device of claim 1, further comprising:
a communicator,
wherein the one or more computer programs further include computer-executable instructions that, when executed by the one or more processors, cause the electronic device to:
based on identifying that the user is an unregistered user as a result that the user authentication was performed, maintain locking of the electronic device, and
control the communicator to transmit a message for guiding that authentication by an unregistered user was performed to an external device connected to the electronic device.

11. The electronic device of claim 1, further comprising:
an outputter,
wherein the one or more computer programs further include computer-executable instructions that, when executed by the one or more processors, cause the electronic device to:
based on identifying that the user is an unregistered user as a result that the user authentication was performed, maintain locking of the electronic device, and
control the outputter to output a guide message for requesting additional authentication.

12. A method of controlling an electronic device, the method comprising:
based on receiving a request for user authentication, acquiring a first signal through at least one motion sensor, and acquiring a second signal through at least one biometric sensor;
acquiring motion information indicating a type of a user's motion based on the first signal;
acquiring biometric information indicating a biometric feature of a user according to the motion of the user based on the second signal; and
inputting the motion information and the biometric information into a user authentication model, and performing the user authentication according to whether the type of the motion and the biometric feature match authentication information.

13. The method of an electronic device of claim 12, further comprising:
based on identifying that the user is a registered user as a result that the user authentication was performed, releasing locking of the electronic device; and
updating the authentication information based on the motion information and the biometric information.

14. The method of an electronic device of claim 12, wherein the acquiring of the second signal comprises:
based on the first signal starting to be received through the at least one motion sensor, acquiring a second signal through the at least one biometric sensor.

15. The method of an electronic device of claim 12,
wherein the at least one biometric sensor comprises a bone conduction sensor, and
wherein the biometric information comprises information on an audio feature transmitted from at least one of a bone or a joint of the user according to the motion of the user.

16. The method of claim 12, further comprising:
performing pre-processing for removing at least one of noises or trends of the first signal and the second signal; and
acquiring the motion information and the biometric information based on the pre-processed first signal and the pre-processed second signal.

17. The method of claim 12, further comprising:
based on identifying that the user is an unregistered user as a result that the user authentication was performed, maintaining locking of the electronic device; and
controlling a communicator to transmit a message for guiding that authentication by an unregistered user was performed to an external device connected to the electronic device.

18. The method of claim 12, further comprising:
based on identifying that the user is an unregistered user as a result that the user authentication was performed, maintaining locking of the electronic device; and
controlling an outputter to output a guide message for requesting additional authentication.

19. One or more non-transitory computer-readable storage media storing computer-executable instructions that, when executed by one or more processors of an electronic device, cause the electronic device to perform operations, the operations comprising:
based on receiving a request for user authentication, acquiring a first signal through at least one motion sensor, and acquiring a second signal through at least one biometric sensor;
acquiring motion information indicating a type of a user's motion based on the first signal;
acquiring biometric information indicating a biometric feature of a user according to the motion of the user based on the second signal; and
inputting the motion information and the biometric information into a user authentication model, and performing the user authentication according to whether the type of the motion and the biometric feature match authentication information.

20. The one or more non-transitory computer-readable storage media of claim 19, the operations further comprising:
based on identifying that the user is a registered user as a result that the user authentication was performed, releasing locking of the electronic device; and
updating the authentication information based on the motion information and the biometric information.
